(19) United States
(12) Patent Application Publication
(10) Pub. No.: US 2022/0354995 A1
(43) Pub. Date: Nov. 10, 2022

(54) ARTIFICIAL CARTILAGE

(71) Applicant: DUKE UNIVERSITY, Durham, NC (US)

(72) Inventors: Benjamin J. WILEY, Durham, NC (US); Feichen YANG, Durham, NC (US); Jiacheng ZHAO, Durham, NC (US)

(21) Appl. No.: 17/764,564

(22) PCT Filed: Sep. 27, 2020
(86) PCT No.: PCT/US2020/052980

§ 371 Date: Mar. 29, 2022

Related U.S. Application Data
(60) Provisional application No. 63/033,275, filed on Jun. 2, 2020.
(60) Provisional application No. 62/908,772, filed on Oct. 1, 2019.

(51) Int. Cl. A61L 27/52; A61L 27/20; A61L 27/26; C08L 1/02; C08L 41/00; C08L 29/04; C08L 77/06

(52) U.S. Cl. CPC A61L 27/52; A61L 27/20; A61L 27/26; C08L 1/02; C08L 41/00; C08L 29/04; C08L 77/06; A61L 2400/12; A61L 2430/06; B82Y 5/00

(57) ABSTRACT

Artificial cartilage materials for repair and replacement of cartilage, such as load-bearing and articular cartilage. The artificial cartilage materials can include a hydrogel with an internal polymer support network that impart the hydrogel mechanical properties similar to that of natural cartilage. In some examples, the hydrogels include a cross-linked cellulose network and a double network of polyvinyl alcohol (PVA) and polyacrylamide-methyl propyl sulfonic acid (PAMPS) polymers. The hydrogels may include specific formulations of different polymers to impart mechanical properties that are within a cartilage equivalent range. The artificial cartilage materials may include a porous base that is bonded to the hydrogel for interfacing with surrounding tissues and promoting ingrowth of bone and/or cartilage. Thus, the materials may be well suited for forming a synthetic graft, such as an osteochondral graft, for implantation into a patient's body.

### CROSS REFERENCE TO RELATED APPLICATIONS

This patent application claims priority to U.S. provisional patent application No. 62/908,772, titled “ARTIFICIAL CARTILAGE,” filed on Oct. 1, 2019, and U.S. provisional patent No. 63/033,275, titled “ARTIFICIAL CARTILAGE,” and filed on Jun. 2, 2020. Each of these applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to artificial cartilage materials in implants suitable for repair of cartilage, including specifically polymer network hydrogel materials and various tools, devices, systems, and methods related thereto.

### BACKGROUND

Every year, approximately 900,000 people in the United States suffer from damage to the articular cartilage that lines the ends of the bones, with the knee being most commonly affected. Articular cartilage lesions have a limited intrinsic ability to heal and often engender osteoarthritis. Current strategies for cartilage restoration include bone marrow stimulation (referred to as microfracture techniques), autologous cartilage cell implantation, and osteochondral transplantation. Approximately 50% of patients treated with microfracture techniques require a second surgery after 10 years. Long-term outcome studies (greater than 10 years) show no advantage of cartilage cell implantation over microfracture techniques, as well as high rates of conversion to total knee arthroplasty. In general, these methods typically have high failure rates, prolonged rehabilitation times (greater than 12 months), can be very costly, and show decreasing efficacy in patients older than 40-50 years. Both microfracture techniques and cartilage cell implantation generally require no full-impact activities for 12 months. Thus, these techniques do not produce reliable long-term clinical benefits.

Osteochondral autograft transfer involves taking small osteochondral grafts from the periphery of the femoral condyles and transplanting them to the prepared lesion site with a press-fit technique. Compared to microfracture and cartilage cell implantation techniques, autologous autografts have been shown to enable a quicker return to full impact activities while having lower failure rates. However, there is a limited amount of autograft that can safely be harvested, meaning only small lesions are treatable with Osteochondral autograft transfer involves. Single-plug allografts from human donors may allow transplantation for most any defect size and is one of the most successful cartilage resurfacing techniques. However, the limited supply of fresh allografts and their high cost limits the wider use of this technique. Further, decellularized, shelf-stable allografts have been shown to have high failure rates due to delamination of the articular cartilage in the graft, likely because the collagen in cartilage undergoes enzymatic degradation in the human body.

Focal joint resurfacing with traditional orthopedic materials is being explored as an alternative strategy, but these implants have limited ability to biologically integrate. There are also concerns that these implants may contribute to joint degeneration through abnormal stress and wear. Hydrogels have been explored as a cartilage substitute because, like cartilage, they mostly consist of water and have a very low coefficient of friction. However, conventional hydrogel formulations do not have sufficient strength to serve as a cartilage replacement in a large weight-bearing joint, such as the knee, and lack the ability to integrate with surrounding tissues.

There is a clear need for improved cartilage repair material and methods for treating patients who suffer from cartilage damage, in particular, cost-effective solutions that can immediately restore the mechanical function of cartilage while enabling long-term biologic integration.

### SUMMARY

The materials, methods and devices described herein related generally to synthetic replacements for cartilage, which may be used as durable orthopedic materials to fill chondral or osteochondral defects. The artificial cartilage materials described herein can be used to form implants: (1) having mechanical properties that closely match those of the native tissues, (2) integrate with surrounding tissues, and (3) resist biodegradation in vivo. The artificial cartilage materials described herein may be coated onto or be formed into an implant (e.g., plug) that is integrated with a patient's tissues. The artificial cartilage may exhibit similar or improved properties compared to natural cartilage in compression and tension, and therefore may be expected to have sufficient fatigue-resistance to undergo the millions of cycles of strain experienced over at least 10 years in the human knee.

Although some triple-network hydrogels show some promise (see, e.g., International Patent Application No. PCT/US2018/059563, filed Nov. 7, 2018, published as WO2019094426A1, and entitled “TRIPLE-NETWORK HYDROGEL IMPLANTS FOR REPAIR OF CARTILAGE,” which is herein incorporated by reference in its entirety), there is a clear need for improved cartilage repair material and methods for treating patients who suffer from cartilage damage.

The artificial cartilage compositions described herein can include a hydrogel with a reinforcement network of polymers; the reinforcement network preferably includes cellulose, such as bacterial cellulose. The hydrogel component of the artificial cartilage can retain a high percentage by weight of water and have very low coefficient of friction, similar to that of natural cartilage. The polymer network can increase the hydrogel's compression and tensile strength, durability, and wear properties within predetermined and beneficial ranges, as described herein. In particular embodiments, the reinforcement network may include a cellulose nanofiber network and a cross-linked double PVA-PAMP network. Compared to other hydrogel formulations, the materials described herein can be characterized as having mechanical properties, such as tensile strength and compression strength, similar to that of human cartilage. Thus, unlike other hydrogel formulations, the artificial cartilage materials can have sufficient strength to serve as a cartilage replacement in a large weight-bearing joint, such as the knee. The artificial cartilage may be made of materials that are biocompatible and resistant to enzymatic breakdown. For example, the reinforcement material can include polymer reinforcement materials such as PVA, PAMPS and/or cellulose, which unlike decellularized autographs, do not degrade by enzymatic reactions.

Bacterial cellulose (also referred to herein as bacteriocellulose and microbial cellulose) and may consist of an ultra-fine network of cellulose nanofibers (e.g., 3-8 nm) which are typically highly uniaxially oriented. This type of 3D structure results in a higher crystallinity (60-80%) of bacterial cellulose. Bacterial cellulose is biodegradable and is synthesized by bacteria. The diameter of typical bacterial cellulose fibers is between about 20-100 nm. Bacterial cellulose has high water retention due to being very hydrophilic and having a high surface area to mass ratio.

Any of the network hydrogels described herein can be bonded to a biocompatible base that is configured to integrate with surrounding tissue and/or bone. The biocompatible base may be made of a synthetic polymer or an alloy (e.g., titanium) having a porous structure conducive for ingrowth of bone and/or cartilage. In some embodiments, the porous base simulates subchondral bone to enable bone and/or cartilage in-growth and long-term fixation, thereby allowing the implant to integrate with the biological bone and/or cartilage. The porous base may be bonded to areas of the hydrogel that are to interface with tissues of the patient's body, such bone and/or cartilage. The strength of the bond between the hydrogel and the porous base may be greater than the cartilage-bone interface.

According to some embodiments, the artificial cartilage material includes a hydrogel that includes: a cross-linked cellulose nanofiber network; and a double network PVA-PAMPS, wherein the PVA has a molecular weight of ranging from about 100,000 and about 175,000, wherein the hydrogel has a weight percent of PVA ranging from about 20% and about 40%, and a weight percent of AMPS between about 20% and about 30%. The hydrogel may have a weight percent of PVA ranging from about 30% and about 40%. The cross-linked cellulose nanofiber network can include bacterial cellulose. The hydrogel can have a weight percent of the cross-linked bacterial cellulose nanofiber network between about 15% and about 45%. The hydrogel can further include MBAA, e.g., as a cross-linker for the PAMPS. The hydrogel can have a concentration of MBAA up to about 60 mM. The hydrogel can have a tensile strength ranging from 8.1 MPa to 40.5 MPa. The hydrogel can have a tensile modulus ranging from 58 MPa to 228 MPa. The hydrogel can have a compressive strength ranging from 14 MPa to 59 MPa. The hydrogel can have a compressive modulus ranging from 8.1 MPa to 20.1 MPa. The hydrogel can have a tensile strength ranging from 8.1 MPa to 40.5 MPa, a tensile modulus ranging from 58 MPa to 228 MPa, a compressive strength ranging from 14 MPa to 59 MPa, and a compressive modulus ranging from 8.1 MPa to 20.1 MPa. The artificial cartilage may further include a porous PEEK base bonded to the hydrogel, where the porous PEEK base includes a porous structure configured to promote ingrowth of bone, cartilage, or bone and cartilage therein. A water content of the hydrogel can range from about 45% to 85% by weight. The water content of the hydrogel can range from about 50% to 60% by weight. A surface of the hydrogel can have a higher coefficient of friction than native cartilage. A surface of the hydrogel can have a higher wear resistance than native cartilage.

According to some embodiments, a method of forming an artificial cartilage material includes: forming a hydrogel which includes: a cross-linked cellulose nanofiber network; and a double network PVA-PAMPS, wherein the PVA has a molecular weight of ranging from about 100,000 and about 175,000, wherein the hydrogel has a weight percent of PVA ranging from about 20% and about 40%, and a weight percent of AMPS between about 20% and about 30%. Forming the hydrogel can include: forming a BC-PVA hydrogel by heating a BC hydrogel in a solution including PVA; and forming a BC-PVA-PAMPS hydrogel by heating the BC-PVA hydrogel in a solution including AMPS. The solution including AMPS can further include MBAA crosslinker to crosslink the PVA and PAMPS. The method can further include bonding the hydrogel to a porous PEEK base, the porous PEEK base comprising a porous structure configured to promote ingrowth of bone, cartilage, or bone and cartilage therein.

The hydrogels described herein have a strength and modulus of cartilage in both tension and compression, and exhibit cartilage-equivalent tensile fatigue at 100,000 cycles. These properties may be attained by infiltrating a bacterial cellulose nanofiber network with a PVA-PAMPS double network hydrogel (forming BC-PVA-PAMPS). The bacterial cellulose may provide tensile strength in a manner analogous to collagen in cartilage, while the PAMPS may provide a fixed negative charge and osmotic restoring force similar to the role of aggrecan in cartilage. The hydrogel has the same aggregate modulus and permeability as cartilage, resulting in the same time-dependent deformation under confined compression. The hydrogel is non-cytotoxic, has a coefficient of friction of about 45% lower than cartilage, is about 4.4 times more wear-resistant than a polyvinyl alcohol hydrogel. The properties of this hydrogel make it an excellent candidate material for replacement of damaged cartilage.

These and other features and advantages are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIGS. 1A and 1B illustrate graphs indicating strength and modulus of various materials including a BC-PVA-PAMPS triple network hydrogel as described herein, and other composite materials as compared to cartilage.

FIGS. 1C and 1D are graphs showing an alternative range of the strength and modulus of various materials compared to human cartilage including a BC-PVA-PAMPS triple network hydrogel as described herein.

FIG. 2 is a bar chart indicating a coefficient of friction for a triple network hydrogel compared to cartilage.

FIGS. 3A-3D illustrate graphs indicating mechanical properties of BC-PVA-PAMPS hydrogel materials with different compositions. FIG. 3A illustrates tensile strength of the hydrogel materials. FIG. 3B illustrates tensile modulus of the hydrogel materials. FIG. 3C illustrates compressive strength of the hydrogel materials. FIG. 3D illustrates compressive modulus of the hydrogel materials.

FIG. 4 illustrates graph showing a stress-strain curve of a cartilage equivalent hydrogel compared to that of pig cartilage.

FIG. 5A is a graph comparing the coefficient of friction of cartilage equivalent hydrogels compared to that of cartilage.

FIG. 5B graphically compares the coefficients of friction of PVA, PVA-PAMPS, porcine cartilage, PAMPS-PDMAAm and BC-PVA-PAMPS hydrogels at different sliding speeds.

FIG. 6 illustrates a graph comparing the average wear depth of cartilage equivalent hydrogels with that of cartilage.

FIG. 7A illustrates an example synthetic graft that includes a triple-network hydrogel bonded to a porous PEEK bone scaffold. FIG. 7B illustrates a graph showing measured mechanical properties of the base of the graft in FIG. 7A.

FIG. 8 illustrates an implant area in a rabbit six weeks after implantation of a graft having a triple-network hydrogel and porous PEEK base.

FIG. 9A is a high-resolution x-ray tomography (MicroCT) image of the synthetic graft in a rabbit 12 weeks after implantation.

FIGS. 9B-9D illustrate example methods of using an artificial cartilage material. FIG. 9B illustrates a region of missing bone and/or cartilage, which may be surgically created. FIG. 9C illustrates an example in which an artificial cartilage material added to fill the region of missing bone and/or cartilage. FIG. 9D illustrates an example in which the artificial cartilage material includes a porous outer region (pores not shown to scale or representative density).

FIGS. 9E-9G illustrate another example of a method of using an artificial cartilage material. FIG. 9E illustrates a region of missing bone and/or cartilage, which may be surgically created. FIG. 9F illustrates an example in which an artificial cartilage material added to fill the region of missing bone and/or cartilage. FIG. 9G illustrates an example in which the artificial cartilage material includes a porous outer region (pores not shown to scale or representative density).

FIGS. 10A and 10B illustrate tribological measurement tools used to determine the coefficient of friction of the hydrogel and cartilage samples.

FIG. 11A is a table (Table 6) showing a comparison of the cartilage-relevant properties of the BC-PVA-PAMPS compositions described herein and other proposed gels for collagen replacements.

FIGS. 11B and 11C illustrate the effect of compression of a sample of BC-PVA-PAMPS (under 100 lbs), in FIGS. 11B and 11C, as compared to other proposed artificial cartilage materials such as PAMPS-PDMAAm and PVA (FIG. 11C).

FIGS. 12A and 12B illustrate tensile (FIG. 12A) and compressive (FIG. 12B) stress-strain curves for BC-PVA, BC-PAMPS, and BC-PVA-PAMPS hydrogels.

FIG. 12C illustrates an example of a BC-PVA-PAMPS hydrogel fabrication process.

FIGS. 12D and 12E show Cryo-SEM images of a BC and the BC-PVA-PAMPS hydrogel, respectively.

FIG. 13A shows a strain vs. time graph for confined, uniaxial creep tests on BC-PVA-PAMPS hydrogel and cartilage under 0.04 MPa.

FIG. 13B is a comparison of the coefficient of friction and wear depth of porcine cartilage and the BC-PVA-PAMPS hydrogel.

FIG. 13C illustrates one example of MicroCT images of the BC-PVA-PAMPS hydrogel, porcine cartilage, PVA-PAMPS hydrogel, PAMPS-PDMAAm hydrogel and PVA hydrogel before and after wear testing of 100,000 rounds at 100 mm s⁻¹ in PBS.

FIG. 13D is a graph showing the maximum cyclic tensile stress applied vs. the number of cycles before fracture. Eight samples indicated by arrows did not fail after 100,000 cycles.

FIG. 14 is a MicroCT images of one example of a BC-PVA-PAMPS hydrogel (top row) and a PVA hydrogel (bottom row) before (left column) and after (right column) 1 million cycles of wear in bovine serum against porcine cartilage.

### DETAILED DESCRIPTION

The methods, materials and devices described herein relate generally to artificial cartilage, and particularly those that include a hydrogel reinforced with a cross-linked fiber (e.g., nanofiber) network typically comprising BC-PVA-PAMPS, where BC is bacteriocellulose/cellulose, PVA is poly(vinyl alcohol) and PAMPS is poly-(2-acrylamido-2-methylpropanesulfonic acid. The materials may exhibit mechanical properties, such as tensile strength and modulus, and compressive strength and modulus, which are well-suited for forming synthetic grafts, such as an osteochondral grafts, for implantation into a patient's body. By controlling the ratios (e.g., weight percentage) of the components within a predefined range, as described herein, the material properties may be kept within a specific and critical range of values that are important for compatibility with implantation. The artificial cartilage materials may be used to treat a subject in need, for example, for articular cartilage replacement applications that meet required mechanical strength to withstand high loads of human joints. The artificial cartilage materials provided herein can be used in a body to augment or replace any tissue such as cartilage, muscle, breast tissue, nucleus pulposus of the intervertebral disc, other soft tissue, and interpositional devices that generally serves as a cushion within a joint.

The artificial cartilage materials described herein may include triple network hydrogels, which include a cross-linked fiber network and a double network hydrogel. The cross-linked fiber network may include a bacterial cellulose nanofiber network (BC). One or both of networks of the double network hydrogel may include polyacrylamide-methyl propyl sulfonic acid (PAMPS). In some embodiments, the triple network hydrogels include one or more crosslinkers such as N,N′-methylenebisacrylamide (MBAA). The materials and methods described herein may be distinguished from the triple network hydrogels and related materials described in International Patent Application Pub. No. WO2019094426A1, which is incorporated by reference herein in its entirety.

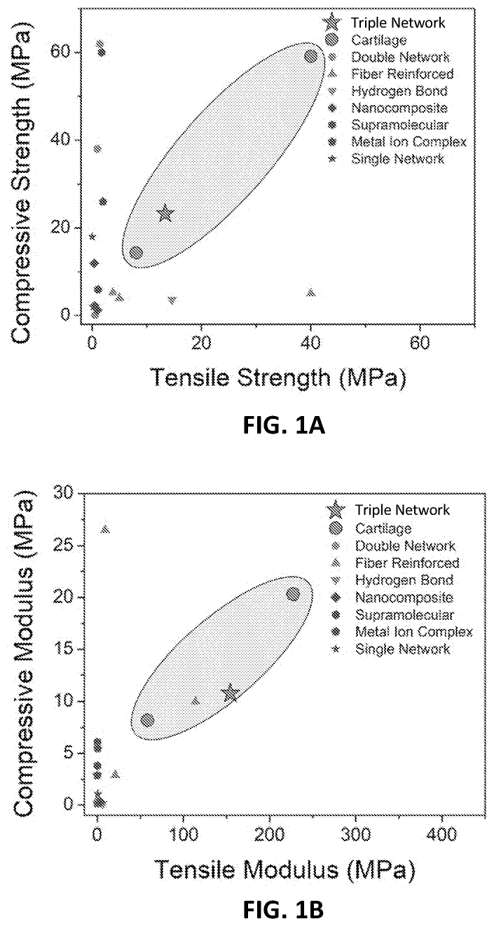

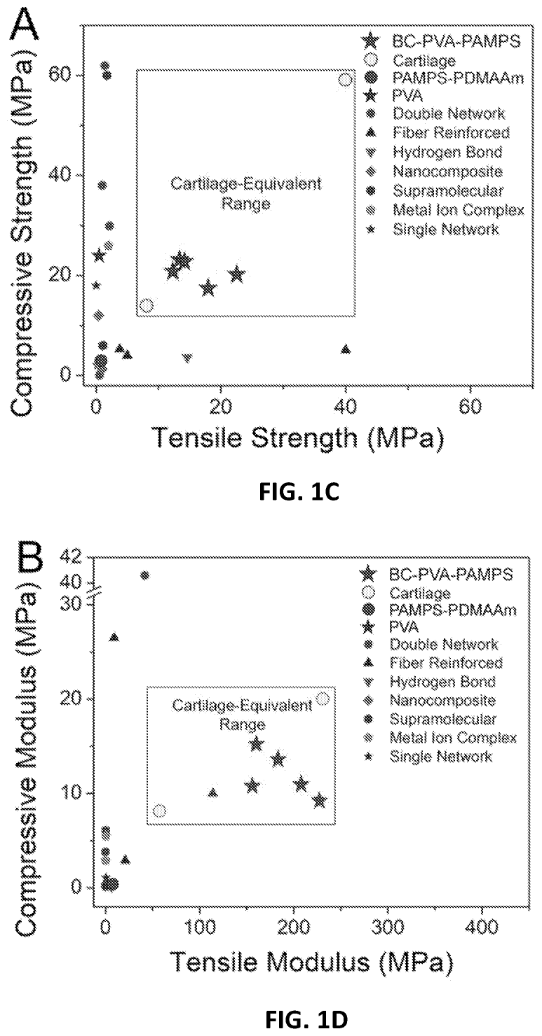

The hydrogels (e.g., the BC-PVA-PAMPS hydrogels) described herein can exhibit mechanical properties that are within a “cartilage equivalent range,” which refers to a range of mechanical properties that are substantially the same as human cartilages. For example, hydrogels that exhibit mechanical properties with a cartilage equivalent range may include those having a tensile strength ranging from 8.1 MPa to 40.5 MPa, a tensile modulus ranging from 58 MPa to 228 MPa, a compressive strength ranging from 14 MPa to 59 MPa, and a compressive modulus ranging from 8.1 MPa to 20.1 MPa. FIGS. 1A and 1B show graphs demonstrating strength and modulus of a triple network hydrogel compared to those of native cartilage and several other hydrogel and composite materials. FIGS. 1C and 1D show alternative ranges (showing the range as a broader rectangular area). Such hydrogels may be referred to herein as “cartilage equivalent hydrogels.” As described herein, the cartilage equivalent hydrogels can exhibit a modulus and strength, under both tension and compression, which are more similar to cartilage compared to the other materials, including other hydrogel formulations. The BC-PVA-PAMPS hydrogels described herein are cartilage-equivalent hydrogels. In FIGS. 1A-1D, the BC-PVA-PAMPS hydrogels are the BC-PVA-PAMPS hydrogels described herein (e.g., having the component ranges as described).

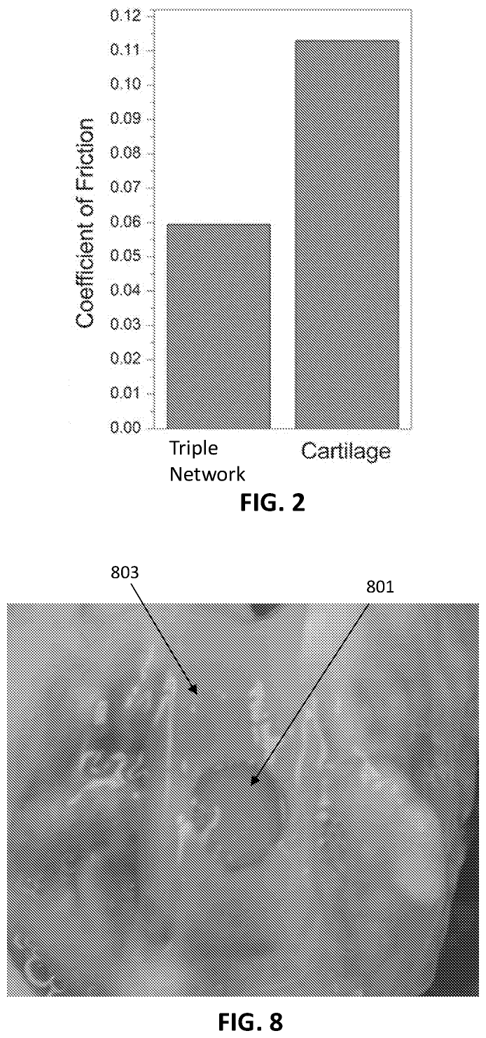

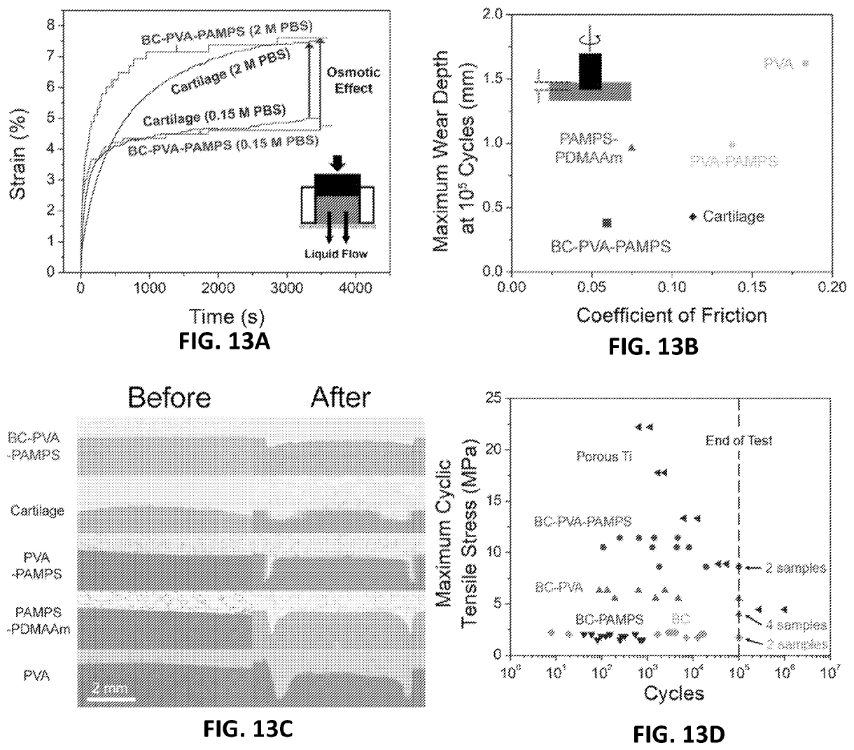

The hydrogels described herein can also exhibit high wear resistance and low coefficient of friction, indicating that the hydrogels can be conducive for long term implantation in a joint of a patient. For example, FIG. 2 shows a bar chart demonstrating the coefficient of friction a triple network hydrogel compared to that of cartilage. Tests were performed with a stainless-steel pin in PBS pressure sensor at a sliding speed of 20 millimeters per second (mm/s). FIG. 2 indicates that the surface of the hydrogel has nearly half the coefficient of friction as cartilage. See also FIG. 13B, described in greater detail below, showing that a wear resistance about the same as cartilage.

The cartilage equivalent hydrogels described herein can include a combination of bacterial cellulose (BC), polyvinyl alcohol (PVA), acrylamido-methylpropane sulfonic acid (AMPS), and optionally N,N′-methylenebisacrylamide (MBAA) as a cross-linking agent. The cartilage equivalent hydrogels described herein may include specific formulations of these components for achieving mechanical properties within the cartilage equivalent range. For example, the improvements described herein can provide an artificial cartilage material that retains its cartilage-similar properties over 10 years-equivalent of typical use, enables bone ingrowth and long-term fixation, and exhibits improved integration with surrounding tissue. To illustrate, a number of hydrogel formulations with associated data are described below.

Table 1 summarizes compositions of example hydrogels having different compositions of BC, polyvinyl alcohol PVA, AMPS, and MBAA. Test were performed on these samples to determine aggregate modulus and fatigue properties of each.

TABLE 1
Sample composition data

| | | PVA | PVA | AMPS | MBAA |
|---|---|---|---|---|---|
| | | Concen- | Molecular | Concen- | Concen- |
| Sample | BC Weight | tration | Weight | tration | tration |
| Number | Fraction | wt. | (g/mol) | wt | (mM) |
| 1 | 13.9% | 40% | 146k | 30% | 60 |
| 2 | 49.8% | 40% | 146k | 30% | 60 |
| 3 | 0 | 40% | 146k | 30% | 60 |
| 4 | 22.1% | 0% | 146k | 30% | 60 |
| 5 | 22.1% | 20% | 146k | 30% | 60 |
| 6 | 22.1% | 40% | 77k | 30% | 60 |
| 7 | 22.1% | 40% | 202k | 30% | 60 |
| 8 | 22.1% | 40% | 146k | 30% | 0 |
| 9 | 22.1% | 40% | 146k | 30% | 20 |
| 10 | 22.1% | 40% | 146k | 30% | 40 |
| 11 | 22.1% | 40% | 146k | 30% | 80 |
| 12 | 22.1% | 40% | 146k | 30% | 100 |
| 13 | 22.1% | 40% | 146k | 0% | 60 |
| 14 | 22.1% | 40% | 146k | 20% | 60 |
| 15 | 22.1% | 40% | 146k | 40% | 60 |
| 16 | 22.1% | 40% | 146k | 30% | 60 |

Table 2 summarizes the final thickness and water content of each of the sample hydrogels in Table 1. The BC film was pressed to different thicknesses to control the initial water content. After autoclaving with PVA and soaking in the AMPS solution, the thickness and water content of the BC-reinforced hydrogel changed due to incorporation of PVA and AMPS solutions.

TABLE 2
Sample thickness and water content

| | Final Hydrogel | Final Hydrogel |
|---|---|---|
| Sample | Thickness | Water Content |
| Number | (mm) | (wt.) |
| 1 | 0.70 | 58.1% |
| 2 | 0.67 | 81.1% |
| 3 | 3.50 | 62.8% |
| 4 | 1.57 | 73.6% |
| 5 | 0.98 | 63.9% |
| 6 | 1.83 | 72.0% |
| 7 | 1.90 | 77.4% |
| 8 | 0.63 | 55.2% |
| 9 | 0.93 | 56.5% |
| 10 | 0.55 | 55.5% |
| 11 | 0.62 | 51.8% |
| 12 | 0.73 | 54.4% |
| 13 | 0.75 | 80.5% |
| 14 | 0.53 | 49.7% |
| 15 | 0.63 | 54.8% |
| 16 | 0.80 | 59.4% |

Table 3 summarizes mechanical properties of each of the sample hydrogels of Table 1. In particular, the tensile strength, tensile modulus, compression strength, and compression modulus were measured.

TABLE 3
Sample mechanical properties data

| | Tensile | Tensile | Compression | Compression |
|---|---|---|---|---|
| Sample | Strength | Modulus | Strength | Modulus |
| Number | (MPa) | (MPa) | (MPa) | (MPa) |
| 1 | 16.49 | 275.83 | 20.49 | 10.86 |
| 2 | 15.90 | 257.40 | 22.73 | 12.14 |
| 3 | 0.85 | | | |
| 4 | 4.08 | 41.27 | | |
| 5 | 8.91 | 122.16 | 7.56 | 11.85 |
| 6 | 5.29 | 52.25 | 11.53 | 9.48 |
| 7 | 3.12 | 32.79 | 2.99 | 7.10 |
| 8 | 14.12 | 158.21 | 22.69 | 15.21 |
| 9 | 12.32 | 181.76 | 20.64 | 13.69 |
| 10 | 17.96 | 226.54 | 17.33 | 9.49 |
| 11 | 21.10 | 357.92 | 22.80 | 13.34 |
| 12 | 15.23 | 266.78 | 26.71 | 11.79 |
| 13 | 11.08 | 115.34 | 10.18 | 6.85 |
| 14 | 22.58 | 206.35 | 20.07 | 10.88 |
| 15 | 18.07 | 267.49 | 12.81 | 9.17 |
| 16 | 13.42 | 155.07 | 23.05 | 10.79 |

The data presented in Tables 1-3 indicate that the hydrogels samples 8, 9, 10, 14 and 16 having a BC weight fraction of 22.1%, a PVA concentration weight percentage of 40%, a PVA molecular weight of 146,000 g/mol, an AMPS concentration weight percentage of 20% or 30%, and a MBAA concentration of 0-60 mM had a tensile strength and modulus and a compression strength and modulus that are within the cartilage equivalent range. These results also indicate that MBAA may not be required in some formulations in order to achieve a cartilage equivalent hydrogel. It should be noted that hydrogels having different BC, PVA AMPS and MBAA composition combinations that are not included in Tables 1-3 may also provide cartilage equivalent mechanical properties. For example, higher AMPS concentration and/or lower MBAA concentrations may also provide a hydrogel within the cartilage equivalent range.

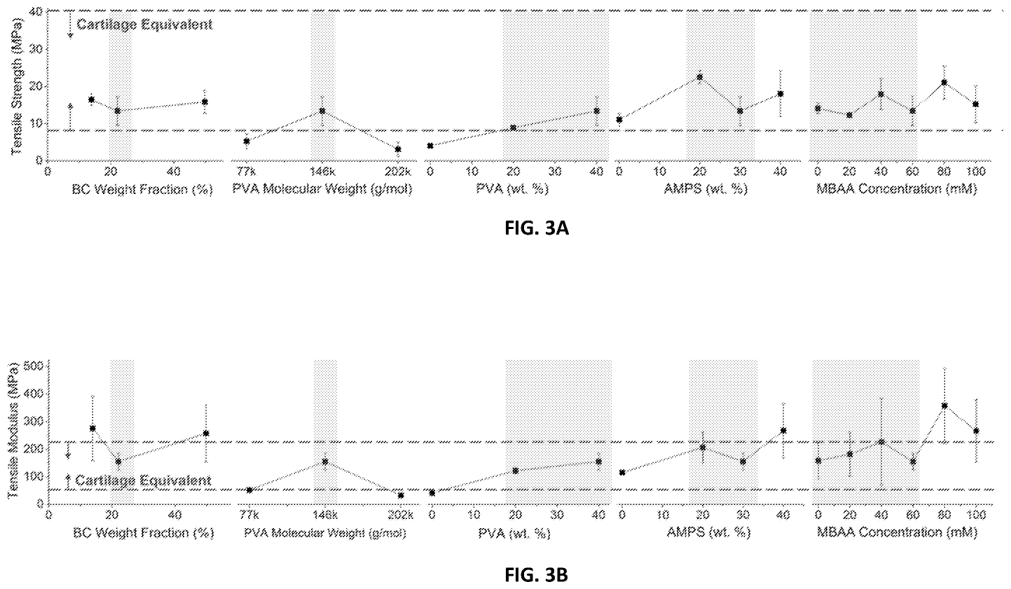

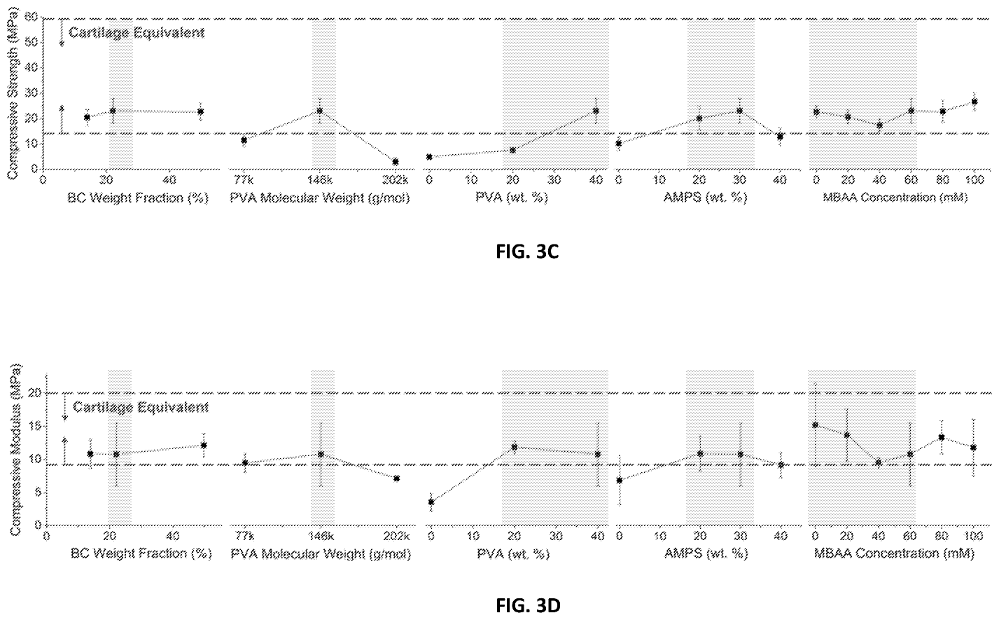

FIGS. 3A-3D show graphs further indicating how different components may effect mechanical properties of the hydrogel. All samples used to collect the data in these graphs were tested with a BC water content of 77.93 wt. %, a PVA with a molecular weight of 146 kDa, a PVA concentration of 40 wt. %, an AMPS concentration of 30% wt., and a MBAA concentration of 60 mM unless stated on horizontal axes. The mechanical properties are presented in comparison to a cartilage equivalent range (indicated with dashed lines). Those sample compositions having results within the cartilage equivalent range are circled. The graphs of FIGS. 3A-3D indicate the effect of BC weight percent, PVA molecular weight, PVA concentration, AMPS concentration, and MBAA concentration as summarized below.

Based on the data provided above, the inventors determined that the BC weight percent is important, in combination with the other components, to achieve the desired and desirable cartilage-like properties. For example, without BC as reinforcement, the tensile strength of the hydrogel was not in a cartilage equivalent range. This result indicates that without BC (e.g., having only PVA and PAMPS), the hydrogel will not have a tensile strength within a cartilage equivalent range. The data also indicates that the range of BC weight percentage values (wt %) was surprising. The BC wt % refers to the weight percentage of BC in the initial BC sheet as determined by drying and weighing the sheet. The sample with 22.1 wt % BC had a slightly lower modulus than the other two, putting it in the cartilage equivalent range. The other samples had tensile modulus values that were too high to be considered cartilage equivalent. From this data, it was found that a BC wt % ranging from about 15% to about 45% can result in a hydrogel having cartilage equivalent mechanical properties. Surprisingly, outside of the range the resulting hydrogel had properties that were not within the effective range. There was a non-linear range of the BC wt % in relation to the cartilage equivalent properties which was not apparent nor expected.

Similarly and related, the PVA molecular weight percentage was found to be important. Moving from a PVA molecular weight of 77,000 to 144,000 g/mol increased the tensile and compressive strength of the hydrogel from outside to inside the cartilage equivalent range. This higher molecular weight polymer leads to increase hydrogen bonding and entanglement between the chains, increasing strength. However, and surprisingly, increasing the molecular weight further to 202 k led to a decrease in strength, making those samples too weak to serve as synthetic cartilage. The decrease in strength may be due to the fact that the higher molecular weight polymer did not fully dissolve fully (e.g., in the autoclave), and was more viscous, leading to a decrease in the incorporation of the PVA into the BC sheet. From this data, a PVA molecular weight ranging from about 100,000 to about 175,000 can result in a hydrogel having cartilage equivalent mechanical properties.

The PVA concentration range was also, in relation to the BC and the PVA molecular weight, found to be important. Without PVA, the tensile strength of the hydrogel was below the cartilage equivalent range, so compression tests were not carried out on those samples. Increasing the PVA content from 20 wt % to 40 wt % lead to an increase in strength, but only the 40 wt % sample had cartilage equivalent compression strength. Higher PVA concentrations did not fully dissolve (e.g., in the autoclave), so 40 wt % may be close to the upper limit of the amount of PVA that may be incorporated into the hydrogel. From this data, a PVA concentration ranging from about 20% to about 40% (in some cases from about 30% to about 40%) by weight can result in a hydrogel having cartilage equivalent mechanical properties, when used in conjunction with BC and specified range of PVA molecular weight.

Further, the AMPS concentration (in the context of the BC and PVA ranges discussed above) was surprisingly important. PAMPS forms a relatively stiff, yet brittle hydrogel. Thus, increasing the AMPS concentration increased the tensile and compressive modulus. Without AMPS, the compression modulus of the hydrogel is too low. Too high of an AMPS concentration (e.g. 40%) made the hydrogel brittle under compression. Therefore, an intermediate range of AMPS (e.g., 20-30 wt %) was found to provide cartilage equivalent mechanical properties. From this data, an AMPS concentration ranging from about 20% to about 30% can result in a hydrogel having cartilage equivalent mechanical properties.

In some variations the MBAA concentration was also examined. MBAA cross-links the PAMPS hydrogel. The MBAA concentration had a relatively minor effect on the properties of the hydrogel, and no MBAA was necessary to provide cartilage equivalent mechanical properties. An MBAA concentration of 80 mM or higher made the hydrogel modulus too high in tension to be cartilage equivalent. From this data, a MBAA concentration ranging from about 0 mM to about 60 mM can result in a hydrogel having cartilage equivalent mechanical properties.

Table 4 summaries compositions that may result in hydrogels having cartilage equivalent properties based on the data presented in Tables 1-3 and FIGS. 3A-3D.

TABLE 4
Composition variable that lead to cartilage equivalent properties

| | PVA | PVA | AMPS | MBAA |
|---|---|---|---|---|
| BC | Concen- | Molecular | Concen- | Concen- |
| wt. % | tration | Weight | tration | tration |
| 15-45% | 20-40% wt. | 100,000-175,000 | 20-30% wt. | 0-60 mM |

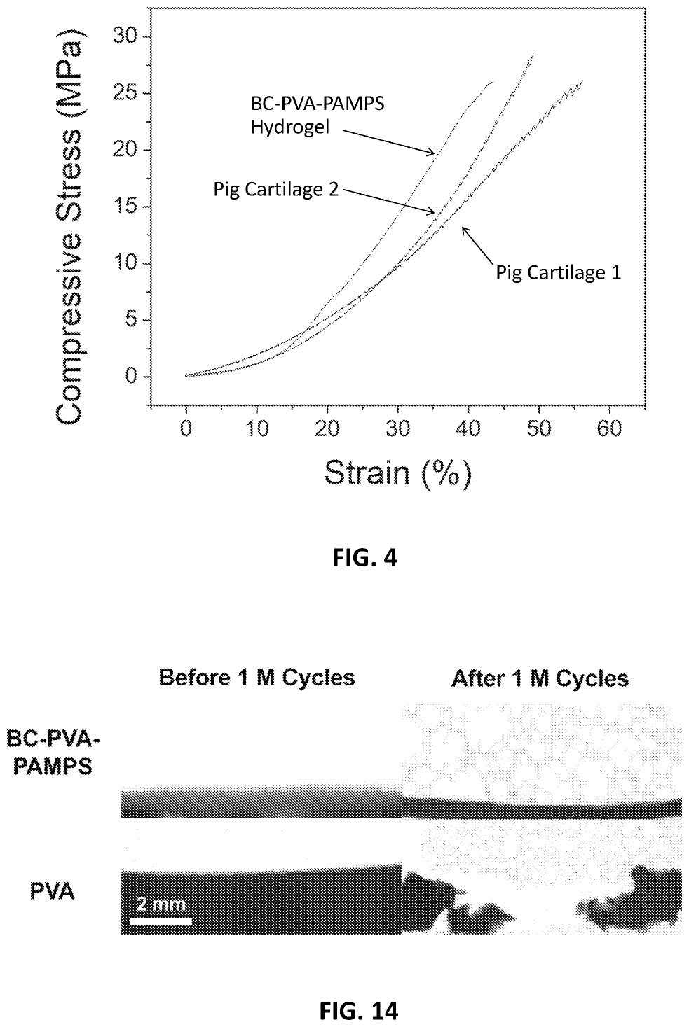

Further, the aggregate modulus of a cartilage equivalent hydrogel was characterized and compared to that of cartilage. FIG. 4 shows a stress-strain curve of a hydrogel having cartilage equivalent mechanical properties (i.e., Sample 16 of Tables 1-3) compared to two different samples of pig cartilage. These results indicate that the cartilage equivalent hydrogel can closely match that of cartilage in unconfined compression, thereby indicating that the aggregate modulus of the cartilage equivalent hydrogel may be similar to cartilage.

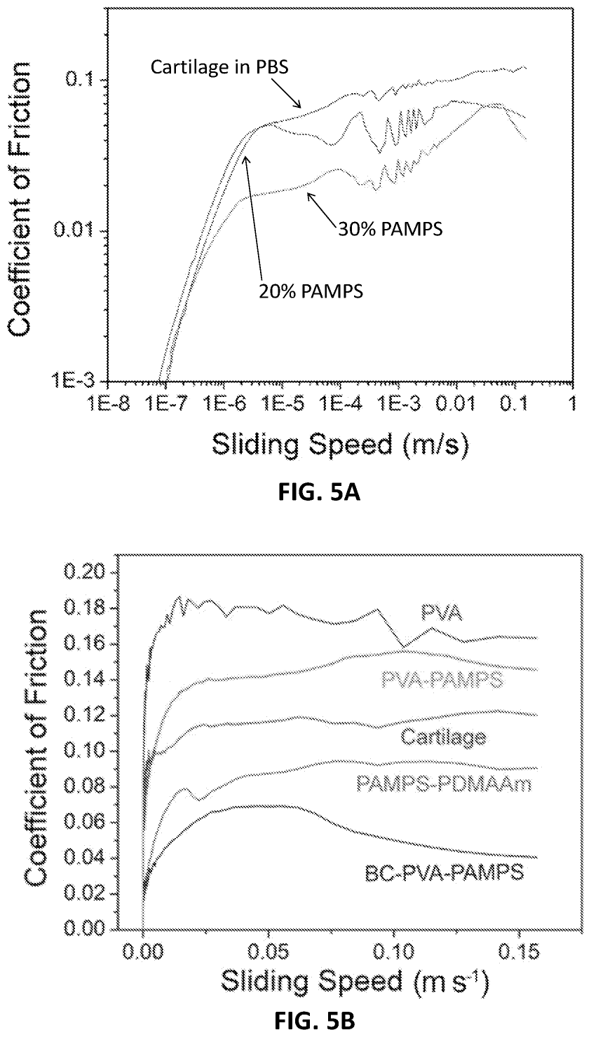

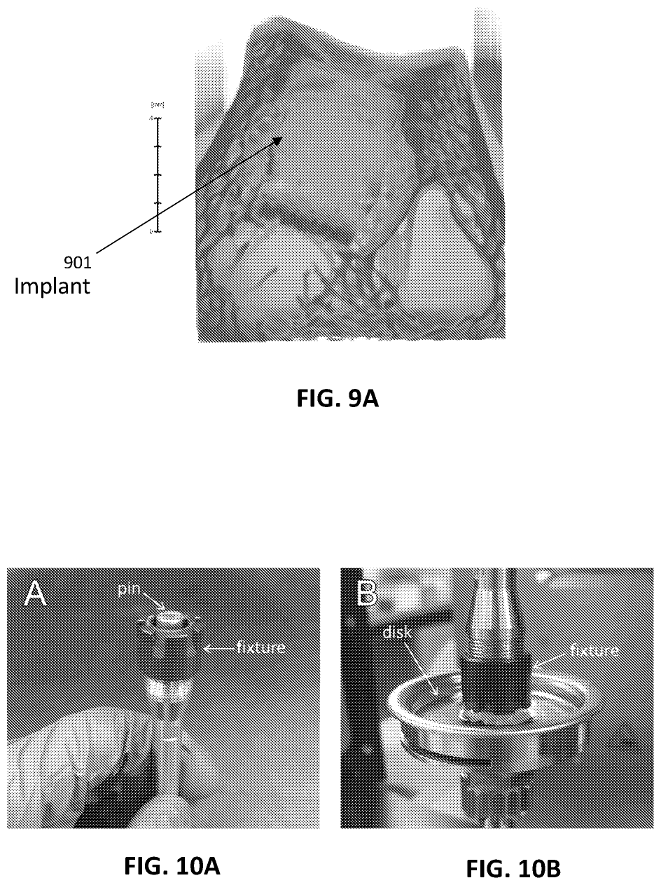

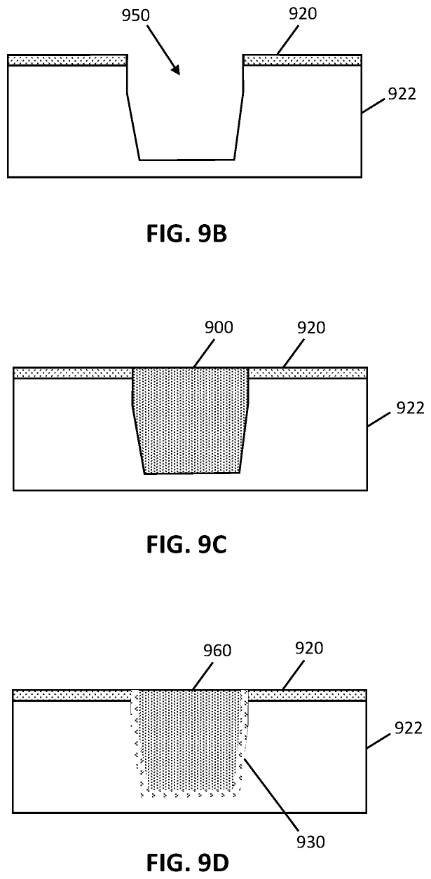

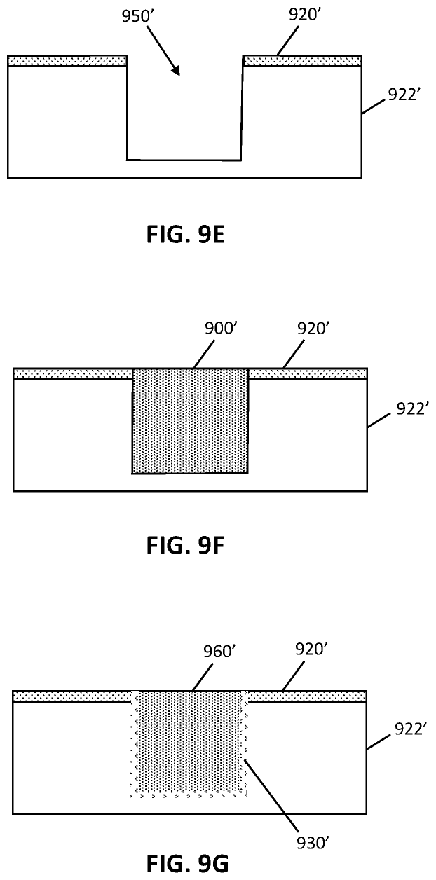

The coefficient of friction of a cartilage equivalent hydrogel was characterized and compared to that of natural cartilage. FIG. 5A shows a graph indicating results from using the example tribological measurement tools and methods (described below with reference to FIGS. 9B and 9C). FIG. 5A compares the coefficient of friction of natural cartilage to that of two cartilage equivalent hydrogel compositions under different sliding speed. The hydrogel samples had an initial BC water content of 77.93 wt %, a PVA with a molecular weight of 146 kDa, a PVA concentration of 40 wt %, an AMPS concentration of 20 or 30 wt %, and an MBAA concentration of 60 mM. Table 5 lists specific coefficient of friction results. FIG. 5B shows similar data, and is discussed in greater detail below.

TABLE 5
Coefficient of friction for cartilage
and cartilage equivalent hydrogels

| Sliding speed | 4 × 10⁻⁵ mm/s | 100 mm/s |
|---|---|---|
| Cartilage | 4.7 × 10⁻⁵ | 0.101 |
| Hydrogel (20% PAMPS) | 8.1 × 10⁻⁵ | 0.046 |
| Hydrogel (30% PAMPS) | 1.2 × 10⁻⁴ | 0.072 |

The results presented in FIG. 5A and Table 5 indicate that the coefficient of friction of the cartilage equivalent hydrogels may be lower than the coefficient of friction of cartilage in a physiologically relevant speed range of 0-100 mm/s.

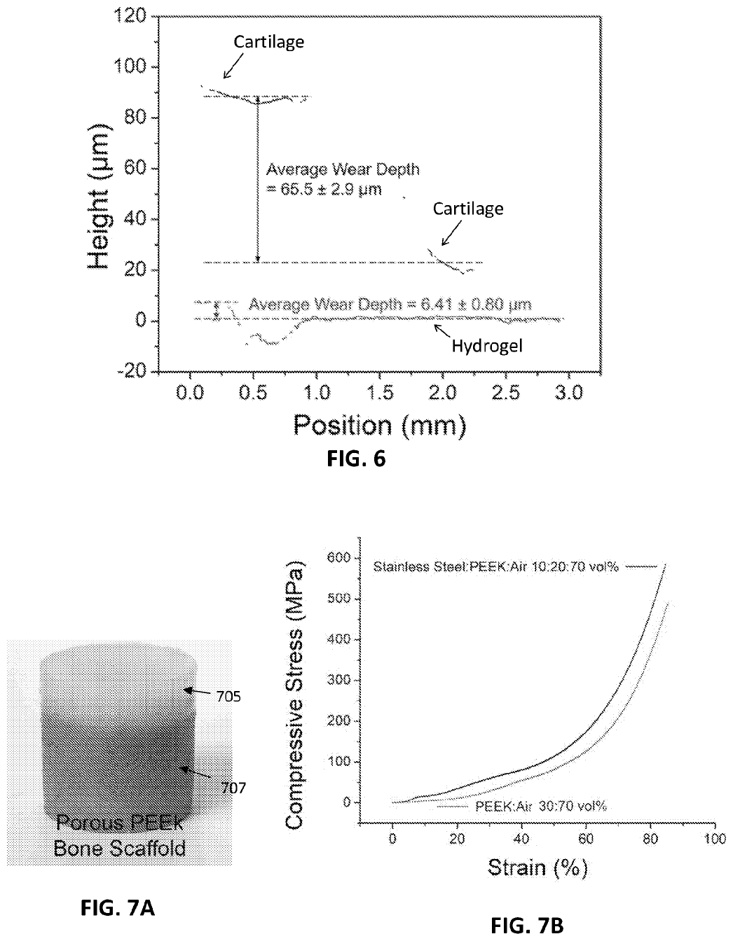

FIG. 6 shows a graph comparing the average wear depth of cartilage equivalent hydrogels with that of cartilage. The wear depth was measured using the example wear test procedure using the tribology equipment described herein. The worn surfaces of the samples were measured using an optical profiler. An average wear depth of the cartilage equivalent hydrogels is 6.41±0.80 micrometers (m), and a wear depth of the cartilage is 65.5±2.9 μm. The results indicate that the cartilage equivalent hydrogels have 10.2 times greater wear resistance than cartilage. It should be noted that under physiological conditions, a cartilage-plug interface may be lubricated by synovial fluid, which may be a superior lubricant than the PBS using during wear testing. Therefore, these results are used to compare the relative wear resistances of the cartilage equivalent hydrogel and the cartilage. That is, the wear resistances may be greater a real case scenario.

### Bonding to a Porous Base

Any of the hydrogel materials described herein can be bonded to a porous base for biologic integration and to ensure long-term attachment of the implant. The porous base may be made of any biocompatible porous polymer that can integrate with surrounding tissue. FIG. 7A shows a graft that includes a porous polyether-ether-ketone (PEEK) bone scaffold 707 bonded to a cartilage equivalent hydrogel 705 (e.g., a BC-PA-PAMPS hydrogel as described herein). The mechanical properties of the graft of FIG. 7A were tested, as shown in FIG. 7B. The porous PEEK base has a strength of 465.9 MPa and a modulus of 11.1 MPa at 1% strain. In comparison, cancellous bone has a strength of 0.51-5.6 MPa and a modulus of 35.84 MPa at 1% strain. To increase the modulus of the porous base to match that of cancellous bone, a stainless steel powder was incorporated with a particle size of 150 μm. This composite has a strength of 533.9 MPa and modulus of 38.1 MPa @ 1% strain. The bone-equivalent modulus and strength that far exceeds that of bone indicate the stainless-steel reinforced porous PEEK base should provide an excellent weight-bearing scaffold for bone ingrowth. The thickness of the hydrogel (e.g., a BC-PA-PAMPS hydrogel as described herein) on scaffold may be about the same, greater or smaller than the thickness of cartilage in the region into which the implant is to be used. For example, the thickness of the BC-PA-PAMPS hydrogel may be between about 0.3 mm and 5 mm (e.g., between about 0.4 mm and 4 mm, between about 0.5 mm and 3 mm, etc.).

In some cases, the bond strength (e.g., shear strength) of the interface between the PEEK base and the hydrogel can be increased by adding one or more bond increasing agents. Such bond increasing agents may include one of various cements that are used to bond tissues, such a calcium phosphate cement, of which there are many commercial examples such as Tetranite™, Cementek®, Biopex®, Rebone®, and Norian®. Alternatives to calcium phosphate cements that may be used with the materials and implants herein may include dental cements, such as Zinc phosphate, Zinc Polycarboxylate, Glass Ionomer, Resin modified glass ionomer, zinc oxide eugenol, resin cements, or calcium hydroxide cements. These cements may increase the shear strength to about 6 MPa or greater. In some cases, the BC is covalently bonded to the porous PEEK scaffold using maleic anhydride or other covalent cross-linking chemicals that lead to the formation of carbon bonds between the BC and the PEEK scaffold. In some embodiments, the BC is grown within and on top of the porous PEEK by culturing Acetobacter xylinum bacteria (e.g., with a Hestrin and Schramm culture medium). The plug can be placed 1 mm below the surface of the static culture medium to ensure integration of the BC with the plug, while maintaining a sufficient thickness of BC for hydrogel reinforcement. The hydrogel can then be polymerized within the BC layer. To facilitate lateral integration of the hydrogel and porous base with surrounding cartilage and bone, the graft can be coated with hydroxyapatite.

FIG. 8 shows an image of an implant area 803 in a rabbit six weeks after implantation of a graft having a hydrogel and porous PEEK base. The image shows that the graft 801 is conforming to the trochlear groove of a rabbit after six weeks. The image also shows that the implant remains intact (e.g., no hydrogel breakdown), the cartilage surrounding the implant appears healthy, and the implant appears to be well-integrated in the defect. There was also no evidence of inflammatory synovitis.

FIG. 9A shows a high-resolution x-ray tomography (MicroCT) image of the synthetic graft 901 in a rabbit 12 weeks after implantation. The graft 901 remains in the defect size and has begun to integrate with surrounding tissues. As described herein, the artificial cartilage materials can be integrated into a patient's body in a number of ways. FIGS. 9B-9D show example methods for using artificial cartilage materials described herein to fill a cavity in a bone and cartilage, according to some embodiments. FIG. 9B shows a cavity 950 formed in a bone 922 and cartilage 920, such as in a bone and cartilage of a joint of a patient. The cavity 950 may be an existing cavity or one that is prepared by a surgeon. FIG. 9C shows an artificial cartilage material 900 configured as a plug that is inserted into the cavity 950. FIG. 9D shows a variation in which a plug-shaped artificial cartilage material 960 includes a porous region 930 (e.g., porous PEEK material). The porous region 930 may be situated on one or more exterior surfaces (e.g., sides) of the artificial cartilage material 960 to improve osseointegration with the bone 922 and/or cartilage 920. The artificial cartilage material plug (e.g., 900 or 960) can be of any shape and size. For instance, the plug can be cylindrical in shape and/or tapered. In some embodiments, the artificial cartilage material plug can be oversized to be elevated from the surrounding cartilage surface 920. In other embodiments, the plug can be undersized to stay recessed in the cavity 950. The over-sizing or under-sizing can be such that the plug can stand proud above the surrounding cartilage surface or recessed from the surrounding cartilage surface by about less than 1 millimeters (mm), by about 1 mm, by more than about 1 mm, by about 2 mm, by about 3 mm, or by about more than 3 mm. In some embodiments the plug can be slightly dehydrated to shrink its size and to allow an easy placement into the cavity. The plug then can be hydrated and swollen in situ to cause a better fit into the cavity. The dehydrated and re-hydrated dimensions of the plug can be tailored to obtain a good fit, under-sizing, or over-sizing of the plug after re-dehydration and re-swelling. The re-dehydration in situ can also be used to increase the friction fit between the plug and the cavity. This can be achieved by tailoring the dimensions and the extent of dehydration such that upon re-dehydration the cross-section of the plug can be larger than the cross-section of the cavity by, for instance, about 1 mm, less than 1 mm, or more than 1 mm. In some embodiments, the cavity is filled with an injectable form of the artificial cartilage material described herein.

FIGS. 9E-9G show another example of the use of an artificial cartilage material as described herein to fill a cavity in a bone and cartilage. FIG. 9E shows a cavity 950′ formed in a bone 922′ and cartilage 920′, such as in a bone and cartilage of a joint of a patient. The cavity 950′ may be an existing cavity or one that is prepared by a surgeon. In this example, the cavity is cylindrical. FIG. 9F shows an artificial cartilage material 900′ configured as a plug that is inserted into the cavity 950′. FIG. 9G shows a variation in which a plug-shaped artificial cartilage material 960′ includes a porous region 930′ (e.g., porous PEEK material). The porous region 930′ may be situated on one or more exterior surfaces (e.g., sides) of the artificial cartilage material 960′ to improve osseointegration with the bone 922′ and/or cartilage 920′. The artificial cartilage material plug (e.g., 900′ or 960′) can be of any shape and size. For instance, the plug can be cylindrical in shape, as shown in FIGS. 9E-9G. In some embodiments, the artificial cartilage material plug can be oversized to be elevated from the surrounding cartilage surface 920′. In other embodiments, the plug can be undersized to stay recessed in the cavity 950′. The over-sizing or under-sizing can be such that the plug can stand proud above the surrounding cartilage surface or recessed from the surrounding cartilage surface by about less than 1 millimeters (mm), by about 1 mm, by more than about 1 mm, by about 2 mm, by about 3 mm, or by about more than 3 mm. In some embodiments the plug can be slightly dehydrated to shrink its size and to allow an easy placement into the cavity. The plug then can be hydrated and swollen in situ to cause a better fit into the cavity. The dehydrated and re-hydrated dimensions of the plug can be tailored to obtain a good fit, under-sizing, or over-sizing of the plug after re-dehydration and re-swelling. The re-dehydration in situ can also be used to increase the friction fit between the plug and the cavity. This can be achieved by tailoring the dimensions and the extent of dehydration such that upon re-dehydration the cross-section of the plug can be larger than the cross-section of the cavity by, for instance, about 1 mm, less than 1 mm, or more than 1 mm. In some embodiments, the cavity is filled with an injectable form of the artificial cartilage material described herein

### Example Hydrogel Preparation

A process for forming a hydrogel includes the following example procedures. Teflon spacers (e.g., 0.25, 0.5, or 1 mm thick) and two aluminum alloy plates are used to sandwich bacterial cellulose (BC) sheets and compress them with a hydraulic press at 500 psi for 5 minutes. The pressed BC is cut into 50 mm (length) by 15 mm (width) sheets so that they can fit into 55 mm (height) by 35 mm (depth) Teflon liners. Eight (8) grams (g) of polyvinyl alcohol (PVA), 12 g of deionized water, and the already cut BC sheets are added into the Teflon liner. The hydrothermal autoclave reactor is securely tighten with the Teflon liner in it. The autoclave reactor is heated to 135° C. and the temperature is maintained for 24 hours. The autoclave reactor is taken out and the cap is carefully open. The BC-PVA sheets are taken out from the liner and the PVA adhered to the surface is remove. The sheets in between 2 glass slides are placed with 0.5 mm spacers fixed to the glass using small clips. The sheets (121° C., 20 minutes) are autoclaved while sandwiched between the glass slides. The sheets are then frozen at −78° C. for 20 minutes and thawed at room temperature (21° C.). A 30 wt % AMPS solution is prepared with 60 mM MBAA crosslinker, 5 mg/ml I-2959 UV-initiator, 0.5 mg/ml potassium persulfate (KPS) heat-initiator. For example, a 30 ml solution was prepared with 270 mg MBAA crosslinker, 150 mg I-2959 UV-initiator, 15 mg KPS heat-initiator and 30 ml 30 wt % AMPS solution. Alternative initiators that may be used include ammonium persulfate, 2,2′-Azobis(2-methylpropionitrile), Benzoyl peroxide, 4,4′-Azobis(4-cyanopentanoic acid), 2,2′-Azodi(2-methylbutyronitrile), Azobis(2,4-dimethyl)valeronitrile, 4,4′-Azobis(4-cyanovaleric acid), Dimethyl 2,2′-Azo-bis(2-methylpropionate), 2,2′-Azobis-(2-amidinopropane), 2,2′-Azobis[2-(2-imidazolin-2-yl)-propane] Dihydrochloride, tert-Butyl hydroperoxide, Cumene Hydroperoxide, Di-tert-butyl peroxide and Dicumyl peroxide. The BC-autoclave-pressed PVA hydrogels are swollen in the AMPS solution made in the previous step for 24 hours. The BC-PA-PAMPS hydrogels are taken out and cured with UV light (VWR transilluminator) for 15 minutes on each side. The BC-PA-PAMPS hydrogels are sealed in an air-tight centrifuge tube and place the tube in 60° C. oven for 8 hours. The BC-PA-PAMPS hydrogels are placed in 1×PBS solution (137 mM NaCl, 2.7 mM KCl, 10 mM Na₂HPO₄ and KH₂PO₄, diluted from 10×PBS solution purchased from VWR) for 24 hours.

### Example Tribology Equipment Setup and Measurement

FIGS. 10A and 10B show tribological measurement tools used to determine the coefficient of friction of the hydrogel and cartilage samples. An Anton Paar MCR-302 rheometer was modified with a tribological attachment to conduct the tribology measurements. FIG. 10A shows a stainless steel pin in the measurement tool fixture. The stainless steel pin has a diameter of 6 mm and was cut and polished with #2500 sandpaper. FIG. 10B shows a pin-on-disk measurement set up for the measurement tool. The stainless steel pin was fixed on the top fixture, while pressed on top of a hydrogel or cartilage disk with a diameter of 12.7 mm. The lubricant is 1×PBS solution. The hydrogel samples were polished with #2500 sandpaper while the cartilage disks were used as harvested.

With a constant pressure of 1 MPa, the top pin was spun at various speeds, and the torque required to maintain that speed was recorded. The coefficient of friction (COF) was estimated with the following equation:

$$C O F = \frac{3}{2} \cdot \frac{Torque}{Radius \cdot Normal Force}$$

The sliding speed was calculated with the following equation:

In a typical case, the radius is 3 mm, and the normal force is 28.26 N.

For wear measurements, all samples are polished with #2500 sandpaper. The roughness of polished hydrogel sample was measured with an optical profiler as 1.9 μm. The tribometer was used to apply 1 MPa with a 316 stainless steel pin, spin at 50 rpm for 5000 rounds while lubricated by 1×PBS.

### Examples

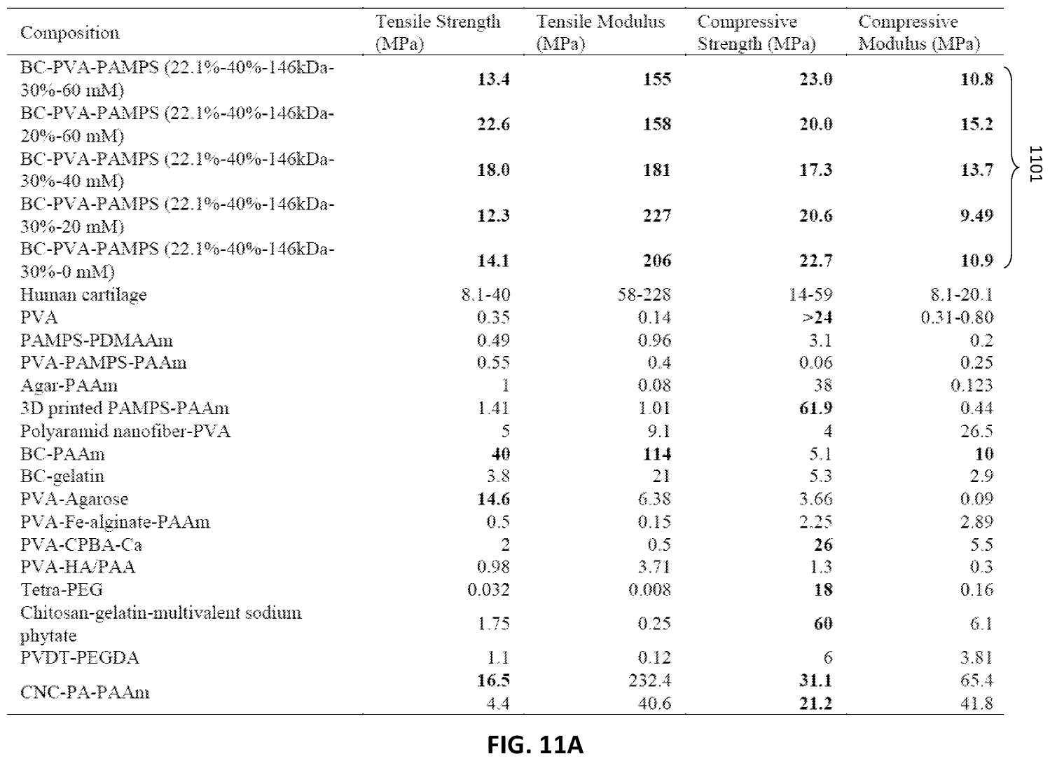

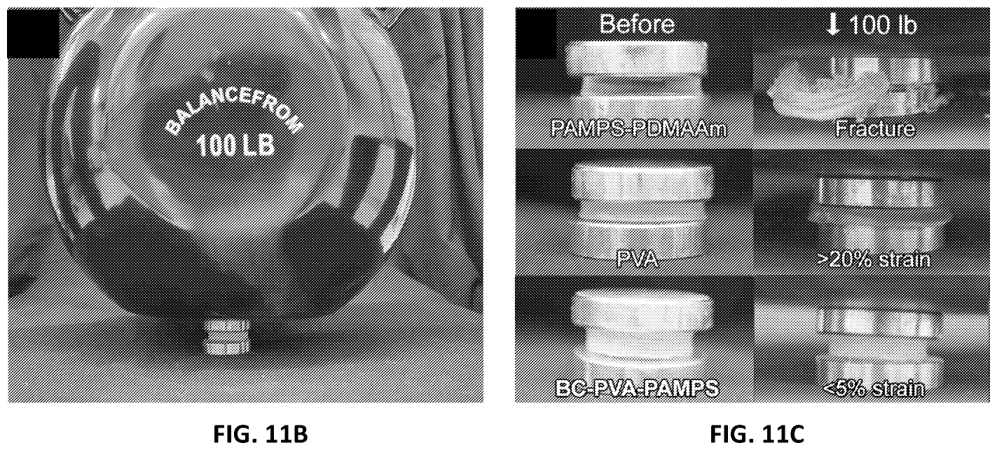

As described above, hydrogels have been extensively explored as a cartilage substitute because, like cartilage, they mostly consist of water and have a low permeability, giving them a very low coefficient of friction (COF). However, current hydrogels do not have sufficient mechanical strength and durability under cyclic loading and wear conditions to serve as a load-bearing cartilage replacement. For example, FIG. 1C (described above) shows that no previously reported gel achieved both the tensile and compressive strength of cartilage. For example, FIG. 11A is a table (table 6) showing examples of known properties of various gel compositions, as compared to the BC-PVA-PAMPS compositions described herein 1101. The five examples of BC-PVA-PAMPS hydrogels 1101 shown in FIG. 11A include a formula of X wt. % BC, Y wt. % PVA, a PVA molecule weight of Z k g mol⁻¹, M wt. % PAMPS and N mM MBAA was denoted as BC-PVA-PAMPS (X %-Y %-Z kDa-M %-N mM). The tensile strength and modulus of PVA and PAMPS-PDMAAm were tested and compared to literature values for the remaining table entries. In table 6 (FIG. 11A), abbreviations used in this table include: BC: bacterial cellulose; PVA: Poly(vinyl alcohol); PAMPS: poly(2-acrylamido-2-methyl-1-propanesulfonic acid sodium salt); PDMAAm: poly(dimethylacrylamide); PAAm: polyacrylamide; CPBA: 4-carboxyphenylboronic acid; HA: hydroxyapatite; PAA: poly(acrylic acid); Tetra-PEG: Tetra-polyethylene glycol; PVDT: poly(2-vinyl-4,6-diamino-1,3,5-triazine); PEGDA: polyethylene glycol diacrylate; CNC: cellulose nanocrystal, PA: phenyl acrylate;

As is shown in FIG. 11A, only the BC-PVA-PAMPS composition had all four metrics (strength and modulus in tension and compression) that fall in the cartilage equivalent range in bold. The bolded values in FIG. 11A fall in the cartilage equivalent range.

If a synthetic hydrogel is to be used for replacement of cartilage, it should have at least the strength of cartilage so that it does not fail during a return to activity, including sporting activities. A hydrogel replacement for cartilage should also have the same time-dependent mechanical properties as cartilage to ensure a normal stress-distribution, as well as a fatigue strength and wear resistance the same as or better than cartilage to ensure durability.

FIG. 11B shows an example of a sample of BC-PVA-PAMPS easy bearing the weight of a 100 lb. kettlebell. In FIG. 11D, cylinders of PAMPS-PDMAAm, PVA, and BC-PVA-PAMPS hydrogel are shown and compared before and after compression with 100 lbs. Only the BC-PAV-PAMPS composition was able to sustain the cartilage-like strength and modulus in tension and compression (bottom right).

As described herein, hydrogels consisting of BC, PVA, and poly(2-acrylamido-2-methyl-1-propanesulfonic acid sodium salt) (PAMPS) are referred to as BC-PVA-PAMPS hydrogels. In FIG. 11C, a cylindrical sample of BC-PVA-PAMPS hydrogel (59% water) with a diameter of 20 mm exhibited <5% strain under a 100 lb. weight (a compressive stress of 1.43 MPa). To put this into context, a 200 lb (890 N) human will have a peak force of 3000 N on the knee during walking, corresponding to a mean contact stress of 2.5 MPa. In comparison, a double network hydrogel consisting of PAMPS and polydimethylacrylamide (PAMPS-PDMAAm) of the same diameter fractured under the 100 lb load even though it has been reported to exhibit a compressive strength of 3.1 MPa. Although the PAMPS-PDMAAm hydrogel has been extensively studied for treatment of cartilage defects, it appears to be too weak to be used in the human knee. A comparison with a polyvinyl alcohol (PVA) hydrogel was also made. PVA hydrogel has received FDA approval to treat arthritis of the first metatarsophalangeal (MTP) joint. As shown in FIG. 11B, PVA hydrogel exhibited significant deformation (>20%) due to its low compressive modulus (0.31-0.8 MPa). Such a large deformation means that PVA alone would transfer stress to the surrounding cartilage and bone if used as synthetic cartilage in the knee. In contrast, the BC-PVA-PAMPS hydrogel has the compressive strength and modulus necessary to potentially serve as a weight-bearing replacement for cartilage.

Without being bound by theory, it is possible that the cartilage-equivalent properties were achieved in the BC-PVA-PAMPS hydrogel by mimicking the structure of cartilage. Articular cartilage principally consists of water (60-85% by weight), collagen fibers (15-22%) with diameters of ˜100 nm, and negatively charged aggrecan (4-7%). The collagen fiber network gives cartilage its high tensile strength. Aggrecan is a brush-like molecule with a negative charge that comes from sulfate groups on the glycosaminoglycan chains attached to a protein core. Aggrecan forms large aggregates with hyaluronan that are trapped within the collagen network, leading to an osmotic pressure that resists compressive loads.

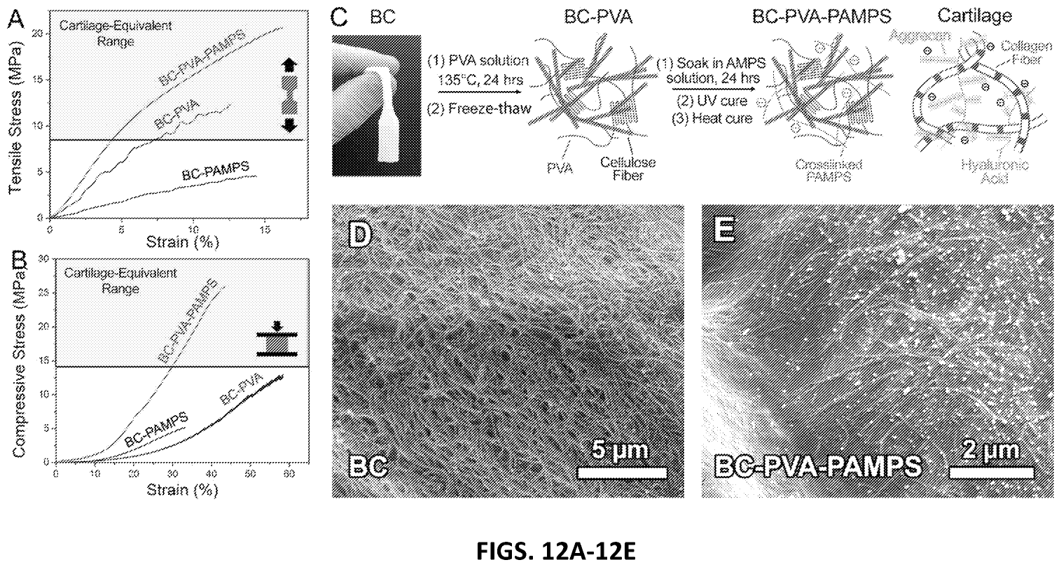

Collagen cannot be used in a synthetic replacement for cartilage because it degrades in the human body, as is demonstrated by the high failure rate of decellularized allografts. Bacterial cellulose (BC) may mimic collagen due to its biocompatibility, high tensile strength, and because the human body lacks the enzymes necessary to degrade cellulose. The second network consisting of a PVA hydrogel was infiltrated into the BC network to provide an elastic restoring force and viscoelastic energy dissipation, and to increase the tensile strength by preventing allowing BC fibers to load share in a composite framework. As shown in FIG. 12A, a BC-PAMPS hydrogel had a tensile strength of 4.6 MPa, lower than the 8.1 MPa required to be in the cartilage-equivalent range. In contrast, a BC-PVA hydrogel has a cartilage-equivalent tensile strength of 12.3 MPa. Adding a PAMPS network to the hydrogel to provides it with a fixed negative charge from the sulfate groups on the PAMPS molecules, which may mimic the role of the chondroitin sulfate and keratan sulfate components that give aggrecan its negative charge. This negative charge may surprisingly results in an osmotic pressure that swells cartilage and contributes to its compressive strength. As shown in FIG. 12B, neither the BC-PAMPS nor the BC-PVA hydrogel had sufficient strength to be considered cartilage-equivalent. By adding the PAMPS network into the BC-PVA hydrogel, both the compressive modulus (23 MPa) and strength (10.8 MPa) were increased to within the cartilage-equivalent range.

FIG. 12C is one example illustrating a fabrication process for BC-PVA-PAMPS hydrogel. In FIG. 12C, a piece of BC may be pressed to a controlled thickness, e.g., 0.5 mm, by using spacers between two metal plates. A cryogenic scanning electron microscopy (cryo-SEM) image, e.g., FIG. 12D, shows the nanofibrous nature of the BC. Next, the pressed BC may be soaked in an aqueous solution (e.g., of 40 wt % PVA at 135° C. for 24 hours) to diffuse the PVA solution into the BC. The BC-PVA gel may then be frozen (e.g., at −78° C. for 30 minutes) and thawed to room temperature to physically crosslink the PVA network. The BC-PVA hydrogel may then be soaked in an AMPS solution (e.g., of 30 wt % AMPS, 60 mM MBAA, 50 mM 12959 and 0.5 mg/mL KPS solution) for 24 hours. The hydrogel may then be cured (e.g., with a UV transilluminator, VWR, e.g., for 15 minutes on each side), and then heat cured in an oven (e.g., at 60° C. for 8 hours) to ensure even and complete curing. The resulting BC-PVA-PAMPS hydrogel may be stored, e.g., in 0.15 M phosphate buffered saline (PBS) solution, for at least 24 hours (or longer). FIG. 12E shows one example of a cryo-SEM image of the surface of a BC-PVA-PAMPS hydrogel.

One example of a BC-PVA-PAMPS hydrogel composition as described herein may include 22.1 wt. % BC, 40 wt. % PVA (molecular weight: 144 k g mol⁻¹), 30 wt. % PAMPS, and 60 mM MBAA. A cartilage-equivalent hydrogel should ideally not only mimic the strength and modulus of cartilage, but also its time-dependent mechanical properties. FIG. 13A shows plots of compressive strain vs. time for BC-PVA-PAMPS and porcine femoral cartilage under confined compression with a constant pressure of 0.04 MPa. The pressure of 0.04 MPa was chosen to keep the strain of the sample in a small range (e.g., <10%). Tests were performed in 0.15 M PBS to mimic the salt concentration in the physiological environment. The creep curve for the BC-PVA-PAMPS hydrogel is similar to porcine cartilage. The aggregate modulus for these samples was determined by fitting the slope of the stress-equilibrium strain curve over the range of 0.04-0.1 MPa. This analysis produced an aggregate modulus of 0.78 MPa for both the BC-PVA-PAMPS hydrogel and porcine cartilage, which is consistent with the range of values reported in the literature for human femoral cartilage (0.46-1.43 MPa). The permeability of BC-PVA-PAMPS hydrogel (3.2×10⁻¹⁵ m⁴ N⁻¹ s⁻¹) is also in the range of values reported for human cartilage (1.2-9.2×10⁻¹⁵ m⁴ N⁻¹ s⁻¹), indicating that the time-dependent deformation of the BC-PVA-PAMPS hydrogel should match that of surrounding cartilage when implanted into a patient's knee.

A similar osmotic effect for the BC-PVA-PAMPS hydrogel on compressive strength was also found. Such an osmotic effect was due to a decrease in the aggregate modulus at a higher salt concentration. As shown in FIG. 13A, the aggregate modulus of the BC-PVA-PAMPS decreased to nearly the same value (0.50 MPa) as porcine cartilage (0.49 MPa) when the PBS concentration was increased to 2.0 M. Thus, a component of the compressive strength and modulus of the BC-PVA-PAMPS hydrogel may be attributed to the osmotic pressure resulting from the large fixed negative charge provided by PAMPS.

Any replacement for cartilage should have a similarly low coefficient of friction (COF) and resistance to wear to ensure that the synthetic replacement is durable and generates minimal wear debris in vivo. A low COF is also desirable to minimize wear of the opposing cartilage surface. The COF of BC-PVA-PAMPS, PVA, PAMPS-PDMAAm, PVA-PAMPS and cartilage samples were tested with a rotating pin-on-fixed disk configuration. The COF of BC-PVA-PAMPS (0.06) was not only the lowest among the hydrogels previously studied for cartilage replacement (0.17 for PVA, 0.08 for PAMPS-PDAAm, 0.13 for PVA-PAMPS), it was also lower than that of porcine articular cartilage (0.11). The low COF may be due, at least in part, to the negative charge of the PAMPS network and the role of BC in reducing the swelling of the hydrogel during soaking in AMPS. The charged surface of the PAMPS hydrogel network can increase the thickness of the water lubrication layer between the gel and the opposing surface, and thereby decrease the COF. Both the PVA-PAMPS and PAMPS-PDMAAm hydrogels have a lower COF than PVA, providing further support for the importance of the negative charge for minimizing the COF. The reason why the BC-PVA-PAMPS hydrogel has a lower COF than PVA-PAMPS is likely because the BC network decreases the volumetric swelling ratio of the hydrogel after being soaked in PBS (160% for BC-PVA-PAMPS hydrogel, 310% for PVA-PAMPS), thus increasing the fixed charge density. The relationship between COF and the sliding speed of PVA, PVA-PAMPS, porcine cartilage, PAMPS-PDMAAm and BC-PVA-PAMPS are shown in FIG. 5B.

The wear resistance of the hydrogels was tested by rotating a 304 stainless-steel pin on top of the samples in 0.15 M PBS for 105 cycles under a pressure of 1 MPa. As shown in FIGS. 13B-13C, the maximum wear depth of BC-PVA-PAMPS hydrogel (370 μm) was 2.6-4.4 times smaller than the other hydrogels (1620 μm, 962 μm and 989 μm for PVA, PAMPS-PDMAAm and PVA-PAMPS hydrogels, respectively). The wear depth for the BC-PVA-PAMPS is even 14% smaller than that of porcine cartilage (429 μm). We attribute this excellent wear resistance to the low COF, high modulus and high strength of the BC-PVA-PAMPS hydrogel. Traditional, more wear-resistant orthopedic materials like cobalt-chromium alloy (CoCr) or ultra-high-molecular-weight polyethylene have a much higher COF (CoCr against cartilage: 0.1-0.2; BC-PVA-PAMPS against cartilage: 0.03) which can lead to wear to the opposing cartilage surface.

The wear of PVA and the BC-PVA-PAMPS hydrogel against cartilage in bovine serum was also measured to determine what amount of wear might be expected under these more physiologically-relevant conditions. This is illustrated in FIG. 14. A cartilage pin was rotated on top of a BC-PVA-PAMPS disk and a PVA disk for 1 million cycles with 1 MPa of pressure. As shown in FIG. 14, the wear of the BC-PVA-PAMPS hydrogel was undetectable under MicroCT, which means that the maximum wear depth was smaller than the resolution of the MicroCT (25 m) after 1 million cycles. On the other hand, the PVA sample was completely worn through (3.5 mm) after 200,000 cycles under the same testing conditions. These results indicate the amount of wear that will occur for the BC-PVA-PAMPS hydrogel in vivo could be negligible.

Cartilage experiences cyclic stress in vivo, so it is important to characterize the fatigue properties of materials that have the potential to be used for cartilage replacement. FIG. 13D shows the results from cyclic tensile testing for the BC-PVA-PAMPS hydrogel, its components in different combinations, as well as porous titanium for comparison. Tensile fatigue failure of collagen may play a role in the mechanical failure of cartilage, and failure in tension may be more clearly defined than failure in compression for cartilage-like materials. Cyclic tests were conducted at 2.5 Hz, so that samples with a higher strength experienced a higher stress rate. The BC-PVA-PAMPS hydrogel exhibited a remarkably high fatigue strength of 8.62 MPa at 105 cycles, which, surprisingly, is equivalent to 85% porous 3D-printed titanium. Addition of PAMPS to BC decreased its resistance to fatigue due to the brittle nature of PAMPS. The addition of PVA to BC increased fatigue resistance due to the toughness of PVA; all four BC-PVA samples were free of damage at 105 cycles. BC-PVA-PAMPS exhibited superior fatigue strength than BC-PAMPS due to the ability of PVA to act as a toughening agent and cancel out the poor fatigue properties of PAMPS. The fatigue strength of BC-PVA-PAMPS is the same as the fatigue strength of cartilage in middle-aged adults.

The BC-PVA-PAMPS hydrogel is biocompatible and exhibits no signs of cell cytotoxicity or lysis, e.g., after incubating L-929 mouse fibroblast cells with an extract of the hydrogel for 48 hours. This result is not surprising given the components of the hydrogel have already been independently demonstrated to be biocompatible. The lack of adverse cell response indicates that this hydrogel may be suitable for use as a cartilage replacement in vivo.

Thus, the BC-PVA-PAMPS hydrogel described herein may have the same strength and modulus as human articular cartilage in compression and tension. Bacterial cellulose nanofibers may provide the hydrogel with a source of tensile strength in a manner analogous to collagen nanofibers in cartilage. PVA may provide an elastic restoring force, viscoelastic energy dissipation, and may prevent stress concentration on individual BC fibers. PAMPS may provide the hydrogel with a source of fixed negative charge and osmotic restoring force similar to the role of aggrecan in cartilage. These BC-PVA-PAMPS hydrogels may also have an aggregate modulus (0.78 MPa) and permeability (3.2×10⁻¹5 m⁴ N⁻¹ s⁻¹) that give it the same time-dependent mechanical response as cartilage under confined compression. BC-PVA-PAMPS hydrogel may exhibit a coefficient of friction (0.06) about half that of cartilage, and may be 4.4 times more resistant to wear than PVA, and exhibited cartilage-equivalent fatigue strength at 100,000 cycles. BC-PVA-PAMPS is not cytotoxic and is comprised of materials that have been previously demonstrated to be biocompatible. Taken together, these properties make the BC-PVA-PAMPS hydrogel an excellent candidate material for use in the repair of cartilage lesions.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. The phrase “between” may be use to describe a range of values and/or positions including the defined end values and/or points. For example, “between 1 and 10,” “between about 1 and 10” or “between about 1 and about 10” may include the values “1” and “10.”

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An artificial cartilage material comprising:
a hydrogel comprising:
a cross-linked cellulose nanofiber network; and
a double network PVA-PAMPS, wherein the PVA has a molecular weight of ranging from about 100,000 and about 175,000, wherein the hydrogel has a weight percent of PVA ranging from about 20% and about 40%, and a weight percent of AMPS between about 20% and about 30%.

2. The artificial cartilage material of claim 1, wherein the hydrogel has a weight percent of PVA ranging from about 30% and about 40%.

3. The artificial cartilage material of claim 1, wherein the cross-linked cellulose nanofiber network comprises bacterial cellulose.

4. The artificial cartilage material of claim 3, wherein the hydrogel has a weight percent of the cross-linked bacterial cellulose nanofiber network between about 15% and about 45%.

5. The artificial cartilage material of claim 1, wherein the hydrogel further comprises MBAA.

6. The artificial cartilage material of claim 5, wherein the hydrogel has a concentration of MBAA up to about 60 mM.

7. The artificial cartilage material of claim 1, wherein the hydrogel has a tensile strength ranging from 8.1 MPa to 40.5 MPa.

8. The artificial cartilage material of claim 1, wherein the hydrogel has a tensile modulus ranging from 58 MPa to 228 MPa.

9. The artificial cartilage material of claim 1, wherein the hydrogel has a compressive strength ranging from 14 MPa to 59 MPa.

10. The artificial cartilage material of claim 1, wherein the hydrogel has a compressive modulus ranging from 8.1 MPa to 20.1 MPa.

11. The artificial cartilage material of claim 1, wherein the hydrogel has a tensile strength ranging from 8.1 MPa to 40.5 MPa, a tensile modulus ranging from 58 MPa to 228 MPa, a compressive strength ranging from 14 MPa to 59 MPa, and a compressive modulus ranging from 8.1 MPa to 20.1 MPa.

12. The artificial cartilage material of claim 1, further comprising a porous PEEK base bonded to the hydrogel, the porous PEEK base comprising a porous structure configured to promote ingrowth of bone, cartilage, or bone and cartilage therein.

13. The artificial cartilage material of claim 1, wherein a water content of the hydrogel ranges from about 45% to 85% by weight.

14. The artificial cartilage material of claim 13, wherein the water content of the hydrogel ranges from about 50% to 60% by weight.

15. The artificial cartilage material of claim 1, wherein a surface of the hydrogel has a higher coefficient of friction than native cartilage.

16. The artificial cartilage material of claim 1, wherein a surface of the hydrogel has a higher wear resistance than native cartilage.

17. A method of forming an artificial cartilage material, the method comprising:
forming a hydrogel comprising:
a cross-linked cellulose nanofiber network; and
a double network PVA-PAMPS, wherein the PVA has a molecular weight of ranging from about 100,000 and about 175,000, wherein the hydrogel has a weight percent of PVA ranging from about 20% and about 40%, and a weight percent of AMPS between about 20% and about 30%.

18. The method of claim 17, wherein forming the hydrogel comprises:
forming a BC-PVA hydrogel by heating a BC hydrogel in a solution comprising PVA; and
forming a BC-PVA-PAMPS hydrogel by heating the BC-PVA hydrogel in a solution comprising AMPS.

19. The method of claim 18, wherein the solution comprising AMPS further comprises MBAA crosslinker to crosslink the PVA and PAMPS.

20. The method of claim 17, wherein further comprising bonding the hydrogel to a porous PEEK base, the porous PEEK base comprising a porous structure configured to promote ingrowth of bone, cartilage, or bone and cartilage therein.
